(19) United States
(12) Patent Application Publication
(10) Pub. No.: US 2022/0295851 A1
(43) Pub. Date: Sep. 22, 2022

(54) Mir Strip

(71) Applicant: Darrien Allen, Highland Park, MI (US)

(72) Inventor: Darrien Allen, Highland Park, MI (US)

(21) Appl. No.: 17/300,135

(22) Filed: Mar. 22, 2021

(51) Int. Cl. A23L 27/00; A23L 33/105

(52) U.S. Cl. CPC A23L 27/79; A23L 33/105; A23V 2002/00

(57) ABSTRACT

An oral dissolving film including a Synsepalum Dulcificum coating. This film may be for the purpose of changing a user's taste buds, by altering the taste of sour or bitter items into a sweet tasting item. Giving the user another alternative option for ingesting the Synsepalum Dulcificum substance for the desired reaction. This alternative option is an easier and safer way for ingesting the Synsepalun Dulcificum substance, compared to the Synsepalum Dulcificum pill form. For the Synsepalum Dulcificum substance to alter a user's taste buds, the substance must be allowed to coat the user's tongue. By coating or infusing an oral dissolving film with Synsepalum Dulcificum it will give a user the ability to place the substance directly on a larger portion of the user's tongue. Creating a safer and more successful way to achieve the desired reaction.

### FIELD OF INVENTION

The present invention Relates to “Oral Dissolving Films”.

### BACKGROUND

Regular Oral Dissolving Films provides quick release of an active Pharmaceutical ingredient. When placed on the tongue of the user. But Regular oral dissolving Films do not contain and distribute a substance that will alter the user's taste buds. User's taste buds will be altered by changing the taste of sour or bitter Foods and Beverages, into sweet tasting foods and beverages.

Presently there is no solution for an Oral dissolving film that contains and distributes a substance that will alter the user's taste buds, by changing Beverages and Foods that are sour or bitter, into sweet tasting Foods and Beverages.

### SUMMARY OF INVENTION

The present invention seeks to provide a solution to this problem. By providing a “Oral Dissolving Film” that contains and distributes a Fruit called “Synsepalum Dulcificum”. When synsepalum dulcificum coats a user's tongue, it alters the user's taste buds. Synsepalum Dulcificum alters taste buds by changing Foods and Beverages. That are sour or bitter, into sweet.

[Basic Description of How it Works]

The Oral Dissolving Film is coated or infused with a Fruit called “Synsepalum Dulcificum”. Synsepalum Dulcificum can be easily applied if you freeze dried and crushed before using. This Fruit can be applied using any sticky agent, preferably “Honey”. Then user places oral dissolving Film on user's tongue. Synsepalum Dulcificum Fruit substance is coated or infused onto the oral dissolving film, giving the user an easy and direct method for placing and distributing the Fruit substance onto a larger area of a user's tongue. User MUST allow the oral dissolving film coated or infused with synsepalum dulcificum to dissolve and coat the user tongue. After the user's tongue is coated. The user's taste buds will be altered for a period of time. User's taste buds will be altered by changing sour or bitter Foods and Beverages, into sweet tasting Foods and Beverages.

Preferably the Oral Dissolving Film is placed directly onto user's tongue. Giving the user an easy and direct way for distributing the synsepalum dulcificum substance onto user's tongue for the desired reaction of altering the user's taste buds, by changing Foods and Beverages that are sour or bitter, into sweet tasting Foods and Beverages.

### DESCRIPTION OF DRAWINGS

FIG. 1. Displays a drawing of a Synsepalum Dulcificum Fruit.

FIG. 2. Displays a drawing of dots representing the Synsepalum Dulcificum Fruit after being freeze dried and crushed.

FIG. 3. Displays a drawing of “Oral Dissolving Film.”

FIG. 4. Displays a drawing of a user's mouth with tongue out.

FIG. 5. Displays a drawing of a user's tongue.

FIG. 6. Displays a drawing of “Oral Dissolving Film” coated with Synsepalum Dulcificum.

FIG. 7. Displays a drawing of a user's tongue coated with Synsepalum Dulcificum. The dots represent the Fruit after being Freeze dried and crushed.

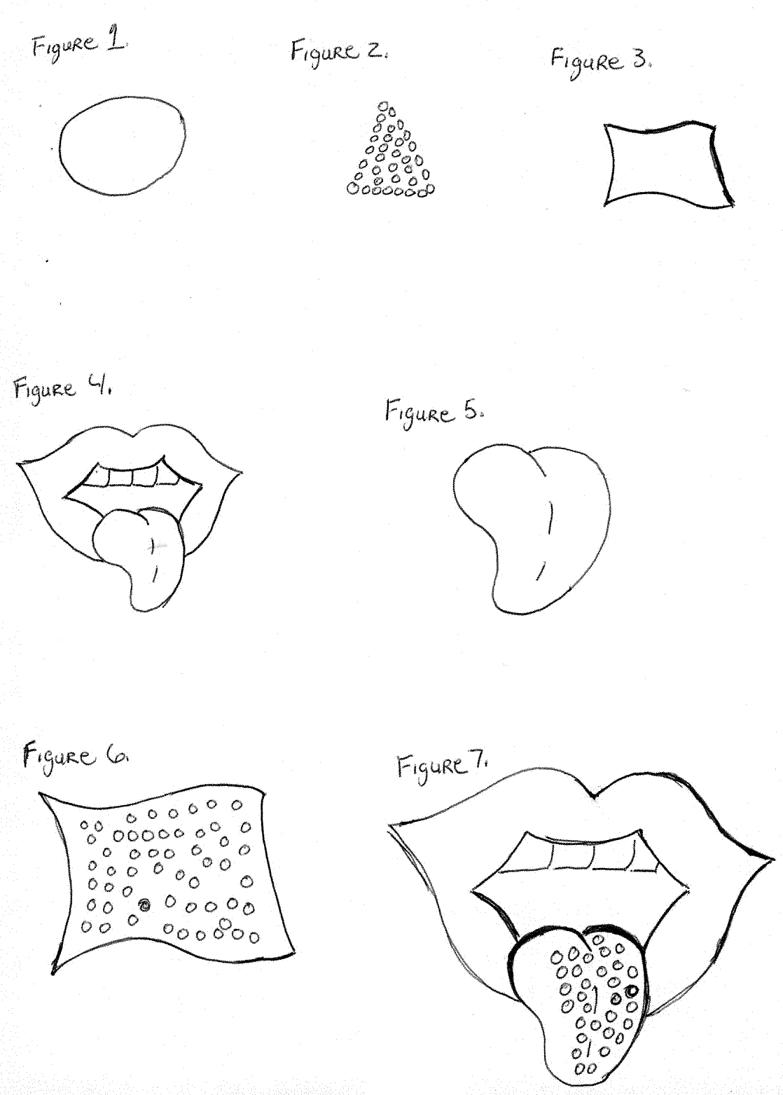

Oral Dissolving Films page 2

The claimed invention is:

1. A oral dissolving film comprising a Synsepalum Dulcificum substance that can be coated or infused into The oral dissolving film.
